(19) United States
(12) Patent Application Publication
(10) Pub. No.: US 2006/0228342 A1
(43) Pub. Date: Oct. 12, 2006

(54) Method for generating antigen-presenting cells

(76) Inventors: Robinson Ramirez-Pineda, Wurzburg (DE); Heidrun Moll, Wurzburg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/515,506

(22) PCT Filed: May 27, 2003
(86) PCT No.: PCT/EP03/05567

§ 371 Date: Sep. 15, 2005

(30) Foreign Application Priority Data
May 28, 2002 (EP) 02011828.7

(51) Int. Cl. A61K 48/00; C12N 5/08; C12N 15/86

(52) U.S. Cl. 424/93.21; 435/372; 435/455

(57) ABSTRACT

Described is a method for the generation of antigen-presenting cells (APC), preferably bone marrow-derived dendritic cells (BMDC) or peripheral blood-derived dendritic cells, as antigen carrier having immunostimulatory properties for anti-infective treatment comprising the steps of (a) pulsing the APC with antigen and (b) treating the APC with a CpG oligonucleotide. Said APC are useful as an immune prophylactic or immune therapeutic agent against diseases like AIDS, tuberculosis, malaria or leishmaniasis.

### FIELD OF THE INVENTION

The present invention relates to a method for the generation of antigen-presenting cells (APC), preferably bone marrow-derived dendritic cells (BMDC) or peripheral blood-derived dendritic cells (DC), as antigen carrier having immunostimulatory properties for anti-infective treatment and cancer vaccination comprising the steps of (a) exposing the APC to antigen and (b) treating the APC with a CpG oligonucleotide. Said APC are useful as an immune prophylactic or immune therapeutic agent against various cancerous and infectious diseases.

### BACKGROUND OF THE INVENTION

In the last century, major advances in vaccination and chemotherapy have accounted for the significant success in prevention and control of a variety of infectious diseases. At the beginning of the 21st century, however, infectious diseases remain to be the first cause of morbidity and mortality in developing countries and are still responsible for a significant proportion of public health problems in the developed world. For example, in 1998, the World Health Organization (WHO) reported that AIDS, tuberculosis, malaria and leishmaniasis caused more than 5 million deaths all over the world, and therefore they keep being a major group of human diseases to be targeted in the future.

Interestingly, these four diseases, although they are different in their origin from cancer, have common characteristics when it comes to a comparison of vaccination strategies that have been explored. In general, positive clinical results are missing in cancer vaccination and the various vaccination strategies have not yet yielded into a therapeutic modality of generally broad applicability producing regressions of metastatic lesions in individual patients. It is remarkable that independent of the goal of vaccination against cancer and infections, some features are in common: 1. no reliably effective vaccines are available, 2. chemotherapy is limited, 3. the causing agent is a transformed cell type or an intracellular pathogen, and 4. although for some of diseased/infected cells antibodies could be beneficial, it is the cellular immune response that is central for mediating protection.

Therefore, the cellular immune responses have been and still are the focus of intense investigations in the last decade, and some of the mechanisms of induction and maintenance have been revealed. Cellular immunity is mediated by CD4⁺ and CD8⁺ T cells which recognize proteins after they have been processed by APC. Their functions are based on phenotypic features and cytokine profiles. Activation of T cells requires the presence of APC, such as B cells and dendritic cells. CD8⁺ T cells recognize antigens presented in the context of MHC class I molecules and their activity is mediated through the production of cytokines such as IFN-gamma and TNF-alpha, and direct cytolytic mechanisms. On the other hand, after recognition of antigens in association with MHC class 11 molecules, CD4⁺ T cells become activated and differentiate into functional subsets termed Th1 and Th2 cells. Th1 cells typically produce IFN-gamma, which is the most important mediator for macrophage activation and the killing of intracellular microorganisms. The induction of IFN-gamma-producing CD4⁺ T cells has been shown to be dependent on the production of IL-12 by APC after exposure to the pathogen at the initiation of the immune response. Thus, in response to many intracellular infections or other pathologic intracellular alterations related to a cancerous cell, IL-12 is the inducer cytokine and IFN-gamma is the effector cytokine. Th2 cells typically produce IL-4, IL-5, IL-6 and IL-10 which stimulate the production of antibodies and are strong inhibitors of the intracellular killing by macrophages.

Recently, a number of reports indicated that there is a striking plasticity in the ability of a given DC subset to respond to different microbes [2-5], suggesting that the type of DC stimulus is a critical factor leading to DC-mediated polarization of the Th cell response

From the experimental murine models of intracellular infections and cancer vaccination and the observations of the human counterparts, it is now widely accepted that the Th1 immune response is protective and especially in infectious diseases Th2 is disease-promoting. This was originally demonstrated for Leishmania major infection in susceptible BALB/c versus resistant C57BL/6 mice but it was also later confirmed to hold true for other bacterial (mycobacteria, Salmonella, Listeria), fungal (Candida, Cryptococcus, Aspergillus, Paracoccidiodes) and some viral (HIV) infections. For this reason, the induction of an effective Th1 immune response seems to be a critical requirement for the development of immune prophylactic or immune therapeutic agents against diseases as diverse in origin as cancer and parasite infection.

According to the findings described above, a prerequisite for the development of vaccines and therapies against intracellular infection and cancer is the preferential induction of the Th1 arm of cellular immune responses. The currently known factors influencing polarization of CD4⁺ T cells include: 1. the local cytokine milieu, 2. the dose and route of antigen administration, 3. the type of APC stimulating the T cell, 4. the “strength” of the signal, i.e. the affinity of the T cell receptor for the MHC-antigen complex plus timing and density of receptor ligation, and, finally, 5. the presence of immunologically active growth factors. From these factors, the cytokine environment surrounding the newly activated T cell seems to be most important.

In the last years, laboratories have been particularly interested in the use of the most potent APC, the dendritic cells (DC), as “natural” adjuvants and potent inducers of a Th1 immune response. For this purpose, the model of murine leishmaniasis was used. It could be shown that after cutaneous infection with Leishmania major, only DC are able to migrate and transport the antigen from the skin to the lymph nodes, and are unique in providing the signals for initiation of the primary specific T-cell response. In addition, DC retain parasite antigen in an immunogenic form for prolonged periods, due to the increased stability of the MHC class II-peptide complexes, and may thus allow the sustained stimulation of parasite-specific T cells that maintain protective immunity against leishmaniasis. These observations prompted the scientists to explore the possibility to use DC as natural adjuvants for immunization against infectious diseases. These studies demonstrated that members of the DC family, epidermal Langerhans cells (LC), after ex vivo pulsing with L. major lysate, can induce long-lasting protection of otherwise susceptible BALB/c mice against subsequent challenges with virulent parasites. This protection was paralleled by a pronounced shift towards a Th1-like pattern, in contrast to the control animals in which a typical Th2 immune response was observed. Thus, one can expect that DC can serve as an effective antigen delivery system for vaccination against infectious diseases and open the possibility for a potential use in therapy. This notion is supported by similar results obtained with other models of bacterial, parasitic, fungal and viral infections.

Availability of DC in sufficient numbers as needed in a therapeutic approach and quality to support clinical treatment of patients will in a long run decide about whether the innate therapeutic potential of DC can be used or not. If this problem is not solved the practical implications will be that DC will stay what they currently are, a powerful research tool. DC constitute a rare but heterogeneous population phenotypically distinct from macrophages (DC are CD14⁻). DC are defined by their potency as APC and are distinct from other well known, but less potent, APC such as B cells and macrophages. DC have been shown to be derived from numerous lineages and dynamically shift their phenotype in response to the local inflammatory environment. The most powerful DC currently known and desirable for use in vaccination approaches are skin-derived DC sometimes referred to as “Langerhans cells” (LC). However, they constitute only 1-3% of the epidermal cells and their isolation from the skin is complicated. Blood DC represent a similarly small population as they contribute to less then 0.3% of the entire circulating blood-leukocyte population. The lack of adequate culture methods for DC is an additional limitation. Thus, in humans, sizable numbers of naturally occurring LC/DC are not accessible.

Other reported sources for DC are less differentiated cells like CD34⁺ progenitors derived from blood monocytes preparations or the uncommitted bone marrow-derived CD34⁺ cells. However, these cells have to be differentiated first ex vivo to acquire a DC phenotype. Human monocyte-derived DC currently represents the easiest accessible source of DC. The number of monocytes available from blood is reasonable and the procedures involved are not too inconvenient for the donor. Generally, DC that have been used in vaccine protocols have been generated from monocytes preparations stimulated with IL-4 and GM-CSF or from monocyte derived precursors (CD34⁺ cells). In humans, monocytes derived cells incubated without IL-4 become activated macrophages. In the murine system, the use of IL-4 is not required to generate DC. In a first step of preparation, peripheral blood mononuclear cells (PBMC) are isolated via density centrifugation. By this way, all red blood cells and the granulocytes are lost in one isolation step. Then the PBMC are cultured for 6 days in the presence of GM-CSF and IL-4. At day 6 the cells have lost CD14 (a monocyte Jineage marker) and gained CD1a. Classical stimuli like lipopolysaccharide (LPS) can stimulate the (immature) DC to produce factors like IL-6, IL-8 and IL-12 (p40 and p70). Despite of limitations posed by the inaccessibility of cutaneous DC they are the best-studied DC type. Much attention has been given to situations in which CD4+ and CD8+ T lymphocytes play a critical role and need to be activated. Cutaneous DC present tumor antigen as well as antigens from infectious agents in the context of class I molecules. Furthermore, LC are able to present exogenous antigens loaded onto class I molecules, a function unique to LC/DC and known as cross-priming. Thus DC can stimulate both T cells and B cells. This finding is of great importance because both the CD4+ and CD8+ T cells are a requirement for protective cancer immunity and need to be activated through class I-presented antigen. In addition to their unique antigen presentation function, cutaneous DC are equipped with extraordinary accessory functions: Together, these exquisite features enable DC to induce primary and secondary immune responses. For this reason, DC are often referred to as “nature's adjuvant” and this opens attractive options in the therapy of cancer and infectious diseases.

In immunotherapy of cancer, the role of cancer-specific CD4+ and CD8+ T cells for generating an antigen-specific and therapeutic immune response is undisputed and a prerequisite for concepts that foresee successful vaccination and are focused to prevention and control of cancer. Nevertheless, due to the lack of immunogenic tumor antigens, the absence of accessory signals and/or active immunosuppression, the natural or vaccination-induced immune response often fails and does not help to combat cancer. Experimental work generated from several laboratories indicates that cutaneous DC present tumor antigens in the context of class I molecules, which is a requirement for the activation of both CD4+ and CD8+ T cells to perform protective cancer immunity. Dendritic cells (DCs) derived from monocytes have been used by few institutions in their current experimental immunotherapy protocols. The results of the studies are difficult to compare since the DC involved have not been generated following a generally accepted standard and their phenotypes are different. The administration of the DC loaded with tumor-associated proteins or peptides resulted in the induction of immune responses against different types of malignant cells. Clinical responses such as stability of disease and tumor regressions have been reported in some patients, particularly with melanoma, myeloma, follicular non-Hodgkin's lymphoma and prostate cancer.

In the clinical trials with DC-based vaccines, a number of important limiting issues have become apparent. These include the optimal source and phenotype of DC, the type of antigen and method of loading DC with antigen, whether to induce differentiation/maturation of DC, the route and timing of immunization, and the appropriate clinical scenario.

The monocyte-derived DC currently used for cancer immunotherapy are not generated following a general standardized scheme. To further explore DC-based approaches, it is therefore very important to establish a protocol for the generation of DC in sufficient amounts and with potent immunostimulatory properties that are similar to those reported for LC (MHC class I-mediated antigen presentation, accessory functions, ability to induce a T heleper response). The use of bone marrow precursor cells seems to be an alternative way to generate larger numbers of DC. However, the same questions arise: what stimuli/culture conditions are required to differentiate them to become the ideal antigen carrier? With respect to shifting and modulating immune responses certain products of bacteria and helminthes stimulate APC and as well DC to prime and activate preferentially Th1 or Th2 cells, respectively. Among the preferred bacterial products are the oligonucleotides that have been shown to be immunostimulators of B cells, NK cells, peripheral blood mononuclear cells (PBMC) and blood dendritic cells (see U.S. Pat. No. 6,429,199, U.S. Pat. No. 6,207,646). In various studies it has been shown by Krieg et al. that an unmethylated cytosine-guanine (CpG) di-nucleotide motif is central for the immunostimulatory property and represented by the general formula: 5′ X₁CG X₂3′; wherein X: is selected from the group consisting of A, G and T; and X₂ is C or T. CpG containing nucleotides have been reported to be in range of 8 to 40 base pairs. However, nucleic acids of any size are immunostimulatory if sufficient immunostimulatory motifs are present The authors (krieg et al.) demonstrate that CpG activates PBMC and that within the various cells types present in monocyte preparations the CpG DNA directly activates the macrophages, which respond with a release of various cytokines (IL-6, GM-CSF and TNF-alpha). Both B cells and NK cells have been shown to be specifically activated by ODN 1668. Krieg et al. furthermore demonstrate that in contrast to monocyte derived dendritic cells it is only the low numbered (0.2%), naturally occurring blood dendritic cell that is susceptible to CpG stimulation. Krieg et al. about write monocyte-derived dendritic cells in U.S. Pat. No. 6,429,199: “DC can be obtained in large numbers, . . . however upon withdrawl of IL-4 lose their DC characterisitcs . . . IL-4 induces Th2 immune response which may not be optimal a specific cytotoxic T cell response; . . . . We found that monocyte-derived dendritic cells are sensitive to LPS but surprisingly are not activated by CpG motifs. It is believed that the inability of monocyte-derived DC to respond to CpG might be due to the unphysiologic methods by which these cells are prepared.” Throughout their work Krieg et al. have profiled the natural occurring DC of the peripheral blood as the prime target of CpG action. However as it has been pointed out before, these physiological DC are because of their limited number not a preferred cell type when it comes to large scale use of therapeutic cells in mammals.

Thus, the technical problem underlying the present invention is to provide in a large scale APC, preferably DC, which can serve as antigen carriers, or natural adjuvant, for anti-cancer and anti-infective treatments.

### SUMMARY OF THE INVENTION

The solution to the above technical problem has been achieved by providing the embodiments characterized by the claims and as follows: It could be demonstrated that particular DC, i.e., BMDC or monocyte-derived DC, can be manipulated in vitro with specific maturation stimuli, i.e., CpG oligonucleotides, resulting in the generation of activated BMDC that exhibit a striking capacity to induce a T cell immune response and protect mammals against an otherwise lethal infection with intracellular pathogens and from cancer.

DC were generated from bone marrow progenitors as described by Lutz et al. and the resulting cell population had a typical DC morphology with a myeloid DC phenotype (MHC class II+, CD80+, CD86+, CD40+, ICAM-1+, CD11c+), and potent MHC class I dependent antigen-presenting functions in allogeneic MLR and in a proliferation assay with Leishmania-specific T hybridoma cells. After 10 days of BMDC culture, the non-adherent cells were collected, resuspended at in culture medium containing GM-CSF and pulsed with antigen.

As a model system, experimental leishmaniasis with Leishmania major was used. A single vaccination of mice for example with DC which had been pulsed in vitro with Leishmania antigen and treated with a CpG oligonucleotide for maturation (DC/CpG/LeishAg) mediated complete protection against subsequent infection with the parasite Leishmania. Control mice which obtained Leishmania antigen or the CpG oligonucleotide alone were not protected. Analysis of the underlying immunological mechanism revealed that vaccination with DC/CpG/LeishAg induced a cell-mediated immune response of the protective type, i.e., an immune response mediated by CD4+ type 1 T helper cells (Th1). The protective effect was stable and long-lasting, i.e., more than 20 weeks after secondary challenge the mice did not exhibit any signs of disease. Using this approach for vaccination against infection or cancer, it can be expected that DC generated from humans or other animals will induce a protective immune response in the treated individual.

### BRIEF DESCRIPTION OF THE DRAWINGS

FIG. 1: Lesion development in BALB/c mice vaccinated with BMDC preparations and infected one week later with L. major parasites

BMDCs were produced and incubated with the different treatments as described in Material and Methods. A and B represent two independent experiments and show the average in footpad swelling for every group ±SEM (n=5).

FIG. 2: Clinical cure of murine cutaneous leishmaniasis induced by CpG-matured lysate-pulsed BMDC is associated with a significant reduction in parasite burden

Control non-vaccinated mice and protected mice from experiment shown in FIG. 1B were sacrificed and the amount of viable parasites was determined by a limiting dilution procedure (A). Samples from footpad suspensions were smeared, stained with Giemsa and observed with light microscopy (B). Pictures displayed are representative from each group.

FIG. 3: The pattern of cytokine expression by lymph node cells from protected mice indicates a shift towards a Th1-like immune response

Pooled lymph node cell suspensions were prepared from some relevant groups shown in FIG. 1B and were incubated for 72 hours in the absence (open) or presence (filled) of Leishmania antigen. Supernatants were assayed for the production of IL-2 (A), IFN-gamma (B) and IL-4 (C) by ELISA.

FIG. 4: The production of Leishmania-specific IgG antibodies in protected mice correlates with a Th1 immune response

Sera from individual mice belonging to the experimental groups shown in FIG. 3 were analyzed for the presence of total IgG (A), IgG1 (B) and IgG2a (C) anti-Leishmania antibodies by ELISA. Results are shown as O.D. and the average is indicated with the bar. The ratio of IgG2a/IgG1 was calculated for each mouse and is shown in D.

FIG. 5: Protection against murine cutaneous leishmaniasis by CPG-matured lysate-pulsed BMDC can be also shown in resistant C57BL/6 mice

BMDC were treated and i.v. injected into mice one week before parasite challenge as described in material and methods. Footpad swelling was then weekly registered (A) and the parasitic load in pooled footpads was qualified after 6 weeks of infection(B).

FIG. 6: (A) Treatment with CpG-matured lysate-pulsed BMDC mediates solid protection against re-infection

Cured mice from the experiment shown in FIG. 1A were re-challenged with 5×10⁵ infective parasites and the lesion development was followed up 20 weekly.

(B) Evaluation of the therapeutic potential of CpG-matured lysate-pulsed BMDC

Mice were infected, i.v. injected with CpG-matured lysate-pulsed BMDC at the time-points indicated in the top of the figure and footpad swelling monitored.

FIG. 7: IL-12 expression by BMDC used for vaccination

BMDC were generated, treated as indicated for 36 hrs and supernatants were separated from cells by centrifugation. Cells were used to amplify the 30 mRNA for IL-12 p40 and IL-12 p35 subunits by RT-PCR, as described in Material and Methods (A). Supernatants were assayed for IL-12 p70 expression by ELISA (B).

### DETAILED DESCRIPTION OF THE INVENTION

In one aspect, the present invention relates to a method for the generation of an APC as antigen carrier having immunostimulatory properties for anti-infective and anti-cancer treatment comprising the following steps:
(a) exposing the APC to antigen (e.g. by pulsing APC with antigen); and
(b) treating the APC with a CpG oligonucleotide.

APC suitable for the method of the present invention comprise different subsets of the DC family with BMDC or peripheral blood-derived DC being preferred. Methods for the generation of DC and the separation of said cells from non-APC are known to the person skilled in the art and described, e.g., in Lutz et al., J. Immunol. Meth. 223: 77-92 (1999); Romani et al., J. Immunol. Meth. 196: 137-151 (1999); Thurner et al., J. Immunol. Meth. 223: 1-15 (1999). Methods for pulsing of the APC in general or specific DC with the antigen are also known to the person skilled in the art and described, e.g., in Flohe et al., Eur. J. Immunol. 28: 3800-3811 (1998) as well as in Example 1(D), below.

The person skilled in the art knows how to carry out treatment of the APC with a CpG oligonucleotide, e.g., by following the instructions given in Example 1(D), below. Steps (a) and (b) can be carried out separately or simultaneously.

Preparation of the CpG oligonucleotide can be carried out according to conventional methods (cf. Sambrook et al., 1989, Molecular Cloning: A Laboratory Manual, 2nd edition, Cold Spring Harbor Laboratory Press, NY, USA).

The term “having immunostimulatory properties” comprises the capability of the matured APC to provide a protective immune response.

Infectious disease related antigens according to this invention is whole cell lysate and antigen mixtures derived from mycobacteria, chlamydia, influenza virus, HPV, HBV, HCV, EBV origin, and molecular defined antigens such as LeIF, elongation factor 4 and LACK from Leishmania, listeriolysin from Listeria monocytogenes and Toxoplasma gondii antigens such as for instance SAG1 and SAG2.

Human cancer antigens recognized by CD8+ t cells are selected from the group of cancer-testis antigens (e.g MAGE-3, BAGE, GAGE, NY-ESO-1), melanocyte differentiation antigens (e.g. Melan-A/Mart-1, tyrosinase, gp100), overexpressed antigens (e.g. Her2/neu, erbB1, p53, MUC-1) and point mutated antigens (e.g. beta-Catenin, MUM-1, CDK4, p53, ras).

In a preferred embodiment of the present invention a not naturally occurring dendritic cell (DC) having specific antigen presentation properties in a mammal comprising a specific disease related antigen and a CpG molecule is generated and used. Said DC derives from CD34+ bone marrow cells precursor cells or peripheral blood monocytes and the APC are BMDC or peripheral blood-monocyte derived DC as antigen carrier having immunostimulatory properties for anti-infective and cancer treatment treatment and the method comprises the following steps:
(a) obtaining bone marrow cells from femurs and/or tibiae or isolating DC precursor cell from peripheral blood monocyte preparations;
(b) culturing the cells under conditions allowing to generate DC;
(c)pulsing the isolated DC with antigen; and
(d) treating the DC with a CpG oligonucleotide.

Methods of the steps (a) and (b) are commonly known and, moreover, described in Example 1(D), below. Steps (c) and (d) are preferably performed simultaneously. If performed sequentially, step (d) is performed before (c).

In a more preferred embodiment of the method of the present invention, the CpG oligonucleotide comprises the nucleic acid sequence 5′-TTCATGACGTTCCTGATGCT-3′. However nucleic acids represented by the general formula(5′X1 CG X2 3′) may be used, wherein X1 is selected from the group consisting of A, G and T; and X2 is C or T as well. A reasonable length for CpG containing nucleotides has been reported to be in range of 8 to 40 base pairs.

The immunogenicity of the antigens used in this invention may be substantially increased by including adjuvants. A preferred embodiment of a vaccine based on the present invention therefore contains QS21, incomplete Freund's adjuvants, IL-2, IL-12, GM-CSF, MPL or an AGP such as RC-529.

In a further more preferred embodiment of the method of the present invention the APC, preferably BMDC, are characterized by their ability to induce a T-helper immune response. This ability can be assayed by standard assays, e.g., the assay described in Example 5, below.

The term “antigen” as used herein comprises a lysate of a pathogenic microorganism, e.g., parasite, (see, for preparation, e.g. Example 1(D), below) or one or more purified proteins of the pathogenic organism. Preferably, the antigen is an isolated protein, or a mixture of such proteins of a microorganism and/or the microorganism is an intracellular pathogen. It is especially preferred that the microbial antigen is selected from the group consisting of (1) cells or an extract, (2) an isolated microbial antigen, (3) an isolated nuclei acid representing the antigen operable linked to a promoter for expressing the isolated antigen, or functional variant thereof, (4) a host cell expressing the isolated polypeptide or a functional variant thereof.

The method of the present invention is useful for providing immune protection against a variety of microorganism, preferably intracellular pathogens (parasites), e.g., HIV, Mycobacterium tuberculosis, Plasmodium, Leishmania, Salmonella, Listeria, Toxoplasma and Chlamydia.

The present invention also relates to APC having immunostimulatory properties, preferably BMDC or peripheral blood-derived DC, which are obtainable by the methods of the present invention described above and exemplified in the Examples, below, as well as a pharmaceutical composition containing said cells, preferably in combination with suitable pharmaceutical carriers. Examples of suitable pharmaceutical carriers are well known in the art and comprise buffered aqueous solutions. Such carriers can be formulated by conventional methods and can be administered to the subject at a suitable dose. Administration of the suitable compositions for vaccination may be effected by different ways, e.g. by intravenous, intraperitoneal, subcutaneous, intramuscular or intradermal administration. The route of administration, of course, depends on the nature of the disease, e.g. kind of pathogen or parasite, and the kind of APC contained in the pharmaceutical composition. The dosage regimen will be determined by the attending physician and other clinical factors. As is well known in the medical arts, dosages for any one patient depends on many factors, including the patient's size, body surface area, age, sex, the particular APC to be administered, time and route of administration, the kind of pathogen, general health and other drugs being administered concurrently.

DC pulsed with antigen and treated with CpG and or pharmaceutical compositions of the present invention may be used as a vaccine. Accordingly, in a further aspect, the present invention relates to a method for inducing an immunological response in a mammal that comprises inoculating the mammal with DC pulsed with antigen and treated with CpG and or pharmaceutical compositions of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering the antigen of the present invention via a vector directing expression of the polynucleotide and coding for the polypeptide in vivo in order to induce such an immunological response to produce cytotoxic and memory T cell or antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use as a vaccine, the DC pulsed with antigen and treated with CpG are normally provided as a vaccine formulation (composition). The formulation may further comprise a suitable carrier. A preferred route for administration is parenterally (for instance, subcutaneous, intra-muscular, intravenous, or intra-dermal injection). Formulations suitable for parenteral administration include aqueous and non-aqueous sterile injection solutions that may contain anti-oxidants, buffers, bacteriostatics and solutes that render the formulation isotonic with the blood of the recipient; and aqueous and non-aqueous sterile suspensions that may include suspending agents or thickening agents.

The packages formulations may be presented in unit-dose or multi-dose/containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. Another way to enhance immunity may require including cytokines and growth factors such as IL-2, IL-4, IL-12, alpha-IFN, GMC-CSF. The dosage will depend on the specific activity of the vaccine and body weight of the recipient and can be readily determined by routine experimentation. Finally, the present invention relates to use of a APC as described above, preferably a BMDC or peripheral blood-derived DC for the preparation of a pharmaceutical composition, preferably an immune prophylactic composition or immune therapeutic composition, for the treatment of a disease caused by an intracellular pathogen. Preferred diseases are AIDS, tuberculosis, malaria, salmonellosis, listeriosis, toxoplasmosis or leishmaniasis.

The following examples explain the invention in more detail.

### EXAMPLE 1

### General Methods

(A) Mice. Female BALB/c and C57BL/6 mice were purchased from Charles River Breeding Laboratories (Sulzfeld, Germany). Animals were 6 to 8 weeks old at the onset of experiments and were kept under conventional conditions.

(B) Parasites and preparation of antigen. Parasites of the Leishmania major isolate MHOM/IL/81/FE/BNI (Solbach et al., Infect. Immun. 54: 909 (1986) were maintained by passage in BALB/c mice and were grown in conventional blood agar plates in vitro. For the preparation of total L. major lysate, stationary-phase promastigotes were collected, washed three times, resuspended at 1×10⁹/ml in PBS and subjected to three cycles of freezing and thawing.

(C) Oligonucleotides. The oligonucleotide 1668 (CpG ODN, 5′ TCCATGACGTTCCTGATGCT 3′) and the control AT-rich oligonucleotide (non-CpG ODN, 5′ ATTATTATTATTATTA TTAT 3′) were synthesized by MWG (Ebersberg, Germany) and were not phosphorothioate-modified.

(D) Preparation and culture of bone marrow-derived dendritic cells (BMDC): Dendritic cells (DC) were generated from bone marrow progenitors using the protocol of Lutz et al., J. Immunol. Meth. 223: 77-92 (1999) with minor modifications. Briefly, total bone marrow cells were obtained from femurs and tibiae after flushing with a syringe containing PBS. Cell suspension was washed and resuspended in culture medium (Click RPMI 1640 supplemented with 10% heat-inactivated fetal calf serum, 2 mM L-glutamine, 10 mM Hepes buffer, 60 μg/ml penicillin, 20 μg/ml gentamycin, 17 mN NaHCO3 and 0.05 mM 2-mercaptoethanol). At day zero, 2×10⁶ cells were seeded in bacteriological petri dishes in a total volume of 10 ml culture medium containing 200 U/ml recombinant murine granulocyte-macrophage colony-stimulating factor (GM-CSF; Peprotek/Tebu, Frankfurt, Germany). Additional 5 ml of culture medium containing 200 U/ml GM-CSF were added at days 3 and 6. After 10 days of culture, non-adherent DC were collected, resuspended at 1×10⁶ cells/ml in fresh culture medium containing 200 U/ml GM-CSF and were incubated overnight (approx. 18 hr) with 30 μl of parasite lysate per ml of culture volume (approx. equivalent to 30 parasites per cell) for antigen pulsing. For some experimental groups, concomitant treatment with recognized inductors of BMDC maturation (lipopolysaccharide, LPS: 1 μg/ml, Sigma, Heidelberg, Germany); CpG and non-CpG ODNs: 25 μg/ml; anti CD40 mAb: 5 μg/ml, Pharmingen, Hamburg; and tumor necrosis factor alpha, TNF-α: 500 U/ml, Peprotec/Tebu, Frankfurt, Germany) was included during pulsing. Control groups with only CpG, non-CpG ODNs and LPS were also included. After overnight incubation, the cells were washed to remove soluble parasite antigen and maturation inductors, and resuspended in PBS for further use.

(E) Treatment of mice: After antigen pulsing/maturation, BMDC were washed and resuspended in PBS, and 5×10⁵ cells were injected intravenously (i.v.) into the tail vein of naive mice. Control mice were injected with PBS. One week later, mice were infected subcutaneously (s.c.) with 2×10⁵ (BALB/c mice) or 2×10⁶ (C57BL/6 mice) stationary-phase L. major promastigotes into the right hind footpad. The course of infection was monitored weekly by measuring the increase in footpad size, compared with the uninfected contralateral footpad. For re-infection experiments, mice were infected with 5×10⁵ parasites into the left hind footpad 9 weeks after the primary infection, which means 3 weeks after complete healing of primary infection. For therapeutic immunization, mice were initially infected and subsequently treated on days 7, 0+7, 7+14 or 14+21 post-infection by i.v. injection with 5×10⁵ BMDCs.

(F) Determination of the parasite load. In order to analyze whether effective leishmanicidal mechanisms were taking place at the site of infection, the amount of viable parasites in the footpads was determined by a limiting dilution technique. Briefly, after 5-6 weeks post infection the right foot was removed, washed with ethanol and rinsed three times with PBS. Preparation of soft tissues was performed by making some slits with a sterile scalpel and by macerating the foot in a cell strainer. Cell suspensions were then passed through a 30G needle in order to assure the release of intracellular parasites. Subsequently, suspensions were centrifuged for 5 minutes at 10g in order to separate tissue clumps and debris. Serial dilutions of the supernatant in 100 μl/well were seeded into 96-well microculture blood-agar plates. For each dilution, replicates of 20 wells were set up. After 10 days of incubation at 28° C. in a humidified atmosphere with 5% CO₂, the cultures were scored for the presence of parasites using an inverted microscope. The estimation of the number of parasites per footpad was done by multiplying the reciprocal of the last dilution showing at least one positive well with the initial dilution factor. For some experimental groups, 10 μl of the footpad cell suspension were smeared onto a glass slide, stained with Giemsa and observed in a conventional light microscope for the presence of L. major amastigotes.

(G) Measurement of cytokine production. Lymph nodes draining the infected footpads were removed 5 weeks after infection. After preparation of single-cell suspensions, 1×10⁶ cells were cultured in 1 ml volume (24-well plates) in the absence or presence of 10 μl of parasite lysate for 72 hours. Thereafter, culture supernatants were harvested for the determination of the cytokines IL-2, IL-4 and IFN-gamma by sandwich ELISA, as published previously (Flohé et al., Eur. J. Immunol. 28:3800-3811 (1998)). IL-12p70 was also measured by sandwich ELISA in supernatants of BMDC cultures after 20 hours of pulsing/maturation.

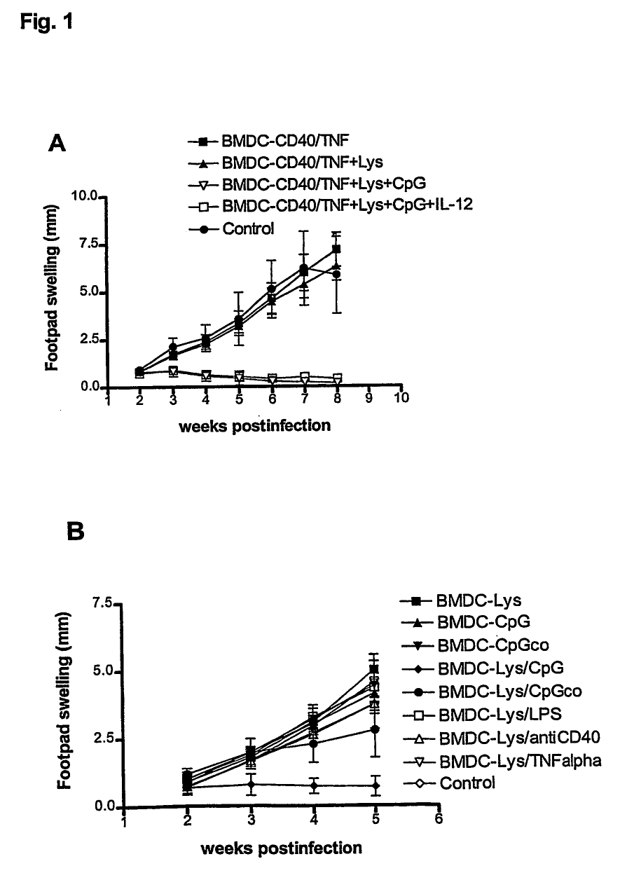

(H) Determination of Leishmania-specific IgG antibodies. Mice of the experiment shown in FIG. 1B were sacrificed 5 weeks after infection and Leishmania-specific IgG, IgG1 and IgG2a serum levels were assayed by ELISA. Plates were coated with total lysate (equivalent to 5×10⁵ parasites/well) and incubated overnight with mouse serum (dilutions: 1:100 for total IgG; 1:50 for IgG1 and IgG2a). For total IgG a second antibody (anti-mouse IgG-alkaline phosphatase conjugate) was incubated for 1 hour and developed with a chromogenic phosphatase substrate. For IgG1 and IgG2a, 1-hour incubation with an isotype-specific second antibody (biotinylated rabbit anti-mouse IgG1 and IgG2a, respectively), 1 hour with streptavidin-conjugated alkaline phosphatase and final substrate development were used. Relative levels of antibodies are presented in optical density (O.D.).

(I) RT-PCR. Total RNA was isolated from BMDC cultures after 36 hours of different pulsing/maturation treatments, using the RNeasy total RNA extraction kit (Qiagen, Hilden, Germany) and 2 μg of RNA were reverse transcribed (Qiagen, Hilden, Germany). Primers for IL-12 p35, IL-12 p40 and β-actin (MWG Biotech, Ebersberg, Germany) were used in a PCR reaction to estimate the relative amount of their respective mRNAs.

### Example 2

### CpG-Matured/Lysate-Pulsed BMDC Protect BALB/c Mice from Cutaneous Leishmaniasis

Recently, it has been reported that Langerhans cells that had been pulsed with Leishmania antigen confer protection against murine leishmaniasis.

Initial attempts to reproduce this protective effect with a different population of DC, the BMDC, were unsuccessful. Several modifications of the protocol with regard to the time of BMDC generation, the amount of BMDC injected into the mice and different conditions of antigen pulsing were performed. However, no protection against infection could be observed (not shown). Thus, additional maturation stimuli of BMDC seemed to be required. Therefore, a series of experiments was performed in which cells were not only pulsed with parasite lysate (as the source of antigen), but in addition treated with inducers of BMDC maturation, including LPS, anti-CD40 antibodies, CpG ODN and TNF-alpha. Two independent and representative experiments are shown in FIG. 1. Again, antigen-pulsed BMDC were not able to induce protection against leishmaniasis (FIG. 1B). When antigen-pulsed BMDC were additionally treated with the maturation inductors LPS, anti-CD40 and TNF-alpha, or combinations of those stimuli, BMDC were also unable to protect against leishmaniasis (FIGS. 1A and 1B). In contrast, immunization of mice with antigen-pulsed BMDC that had been cultured in the presence of CpG ODN conferred complete protection against subsequent infection with L. major in otherwise susceptible BALB/c mice (FIGS. 1A and 1B). All mice that had been vaccinated with those cells developed only a minor footpad swelling (always less than 1 mm, FIG. 1A and 1B), which peaked 3 weeks after infection, and were completely cured after 6 weeks (FIG. 1A). None of the mice in this group showed any sign of ulceration. The course of lesion development in control groups immunized with non-pulsed CpG-treated BMDC or pulsed BMDC treated with a non-CpG motif AT-rich ODN was comparable to the PBS control group (FIG. 1B). These findings demonstrate that a single i.v. injection with antigen-pulsed CpG-matured BMDC mediates complete protection against murine leishmaniasis.

### Example 3

### Clinical Cure Correlates with a Significant Reduction in Parasite Burden

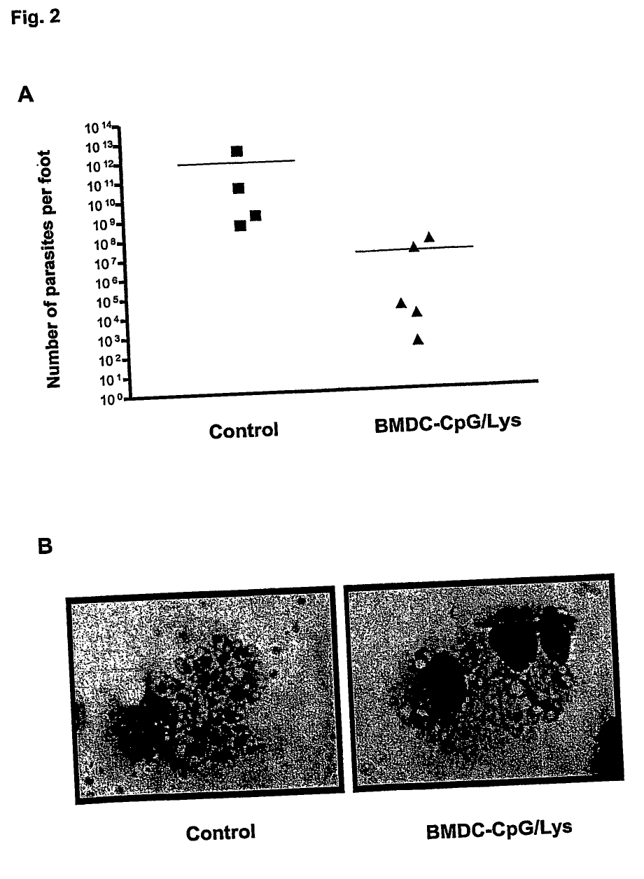

It was analyzed whether the protection induced by CpG-matured antigen-pulsed BMDC is paralleled by an effective control of parasite replication at the site of infection. FIG. 2A shows the parasite loads in individually analyzed mice from the protected and the control groups. All mice that had been vaccinated with CpG/antigen-BMDC had a significantly lower parasite burden than the control mice. On average, there was a more than 10⁴ fold reduction in the number of parasites per footpad (7.3×10¹¹ and 1.2×10⁷ for control and protected groups, respectively). When smears from the control footpads were analyzed under the microscope, an uncountable high amount of parasites was seen and, as shown in FIG. 2B, macrophages were typically full of intracellular parasites, indicating active replication. In contrast, in samples obtained from the protected footpads, parasites could hardly be detected and the typical observation was the presence of no or very few intracellular amastigotes (FIG. 2B). These results indicate that the protection induced by immunization with CpG-matured antigen-pulsed BMDC in susceptible mice is due to an acquired ability to efficiently activate anti-Leishmania effector mechanisms.

### Example 4

### CpG-Matured/Lysate-Pulsed BMDC Induce a Shift in the Cytokine Profile

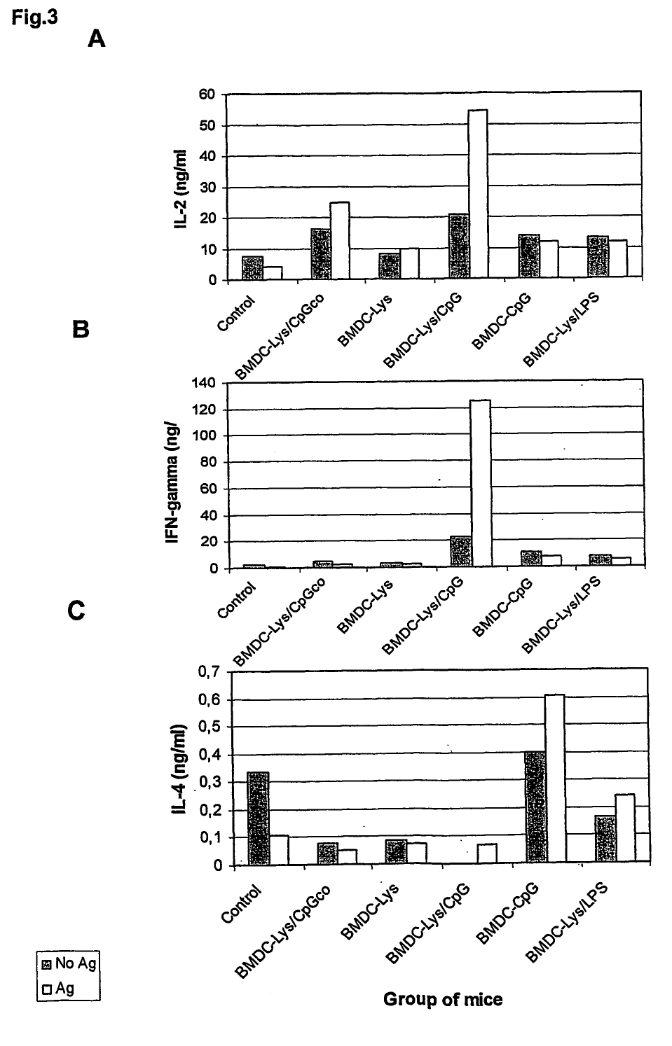

In order to determine whether the protection induced by BMDC is associated with a different profile in cytokine expression, the secretion of IL-2, IFN-gamma and IL-4 by lymph node cells was assessed. Mice from the most relevant experimental groups shown in FIG. 1B were sacrificed 5 weeks after infection, and total lymph node cells were cultured for cytokine analysis by ELISA. The levels of IL-2 in the absence of Leishmania antigen ranged between 7.6 and 20.7 ng/ml with the maximal level exhibited by the protected group that had been vaccinated with BMDC-lysate-CpG. However, this difference was strikingly enhanced when Leishmania antigen was added to the culture. A 13-fold higher level of this cytokine was observed in the protected compared with the control and 2 to 4-fold higher than the other groups (FIG. 3A). An even more pronounced difference was observed when IFN-gamma levels were determined. As shown in FIG. 3B, a 10-fold increase was observed in the absence of antigen when the protected group is compared with the control group and 2 to 7-fold when compared with other groups. When Leishmania antigen was present in the cultures, a 151 and 16 to 60-fold higher level of IFN-gamma was observed when protected group is compared with control and the other groups respectively (FIG. 3B). In contrast to IL-2 and IFN-gamma, lymph node cells from mice belonging to the protected BMDC-Lysate-CpG group secreted no detectable, or very low, levels of IL-4 in the absence or presence of antigen, respectively (FIG. 3C). Some of the non-protected groups were also low IL-4 producers. Thus, in mice treated with CpG-matured/lysate-pulsed BMDC, the cytokine profile induced in lymph node cells was strongly shifted towards Th1-like immune response.

### Example 5

### The Pattern of Leishmania-Specific IgG Antibodies Correlates with the Induction of a Th1 Immune Response in CpG-Matured/Lysate-Pulsed BMDC-Vaccinated Mice

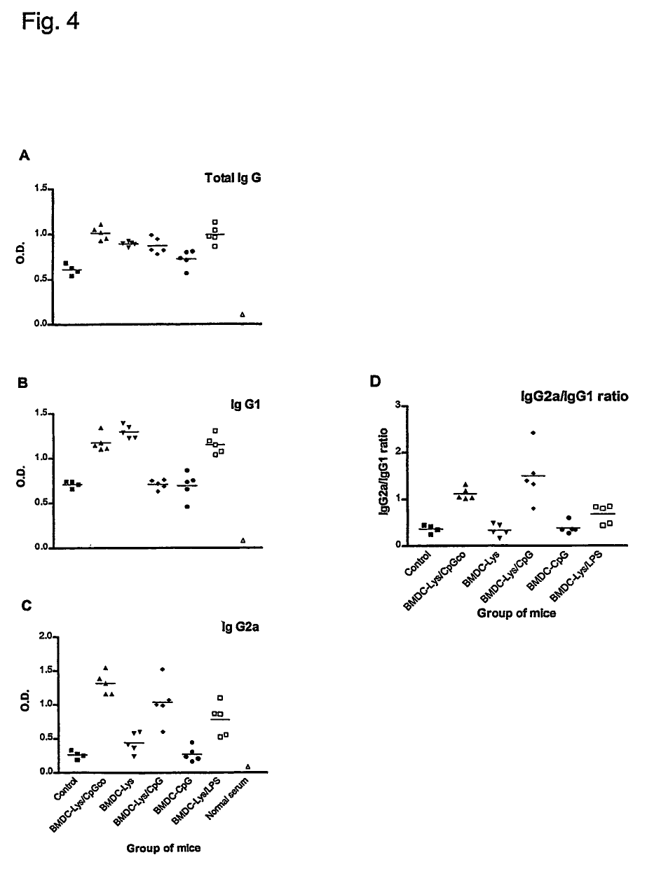

It is well known that different IgG subclass profiles correlate with Th1 or Th2 immune response. The presence of high levels of IgG1 and low titers of IgG2a anti-Leishmania antibodies is associated with a Th2 response and the reverse distribution with a Th1 response. Thus, it was investigated whether the pattern of IgG subclass production was shifted towards the Th1-type response in the protected group. Mice from the most relevant experimental groups showed in FIG. 1B were sacrificed 5 weeks post infection, and the relative levels of total IgG, IgG1 and IgG2a antibodies were determined by ELISA. As shown in FIG. 4A, the levels of Leishmania-specific total IgG antibodies were variable but significant in all experimental groups. When the IgG subclass distribution was determined, a clear tendency to produce low IgG1 and high IgG2a levels was observed in the serum of protected mice that had been treated with BMDC-lysate-CpG (FIGS. 4B and 4C). Some groups showed low levels of IgG1 and some high levels of IgG2a, but only the protective CpG matured/lysate-pulsed BMDC were able to induce the combination of both. A simpler parameter to see Th1-like shifting seems to be the relative ratio of IgG2a to IgG1, with higher values indicating Th1 induction. As showed in FIG. 4D, the protected BMDC-Lysate-CpG group exhibited the highest IgG2a/IgG1 average ratio which was 4 times higher than for the control group (1.4992 and 0.3661, respectively). Some other groups showed higher ratio values than the control group due to higher IgG2a levels, but in contrast to the protected group, they also exhibited higher levels in IgG1 than the control group. Taken together, these results indicate that only the protected group of mice, which was vaccinated with CpG-maturated lysate-pulsed BMDC, produces a pattern of anti-Leishmania antibodies that correlates with the induction of a strong Th1 immune response after infection with virulent L. major.

### Example 6

### The Protective Effect of CpG-Matured/Lysate-Pulsed BMDC is Also Observed in the Resistant Strain of C57BL/6 Mice

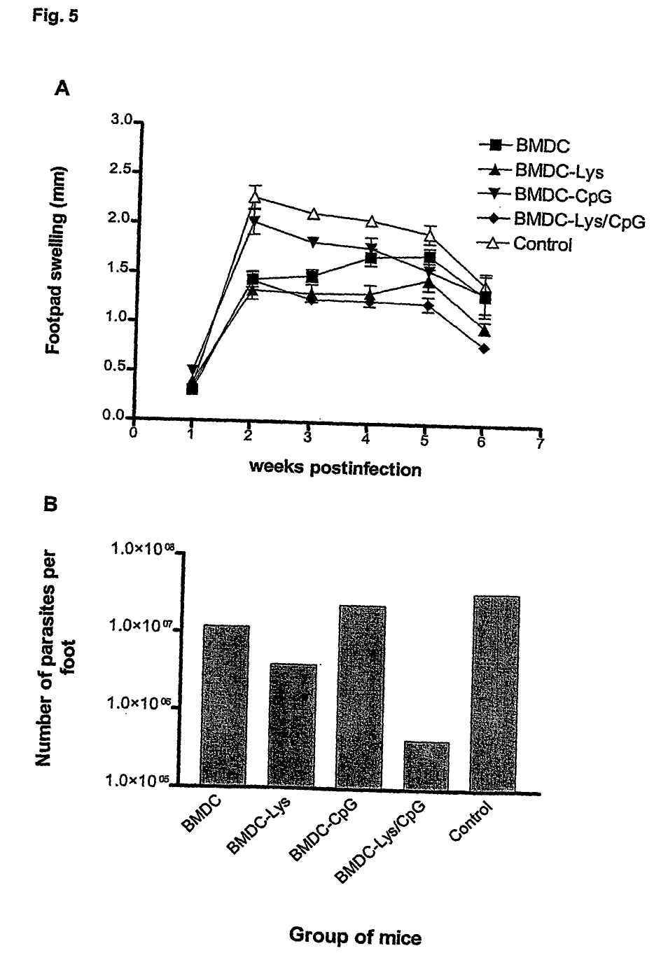

It was investigated whether this approach is also applicable to a different strain of mice which is resistant to L. major infection. As very well known (and shown in FIG. 5A, control group), C57BL/6 mice develop a limited inflammation in the footpad after infection and finally cure after 6-8 weeks of infection. However, when these mice are vaccinated with CpG-matured/lysate-pulsed BMDC one week before the infection, a significant reduction in the footpad swelling is observed, with a lower maximal peak and faster healing (FIG. 5A). When these mice are vaccinated with BMDC alone, an initial unspecific effect is observed. However, these mice reached a footpad swelling comparable to the control after 4-5 weeks post-infection (FIG. 5A). As expected, vaccination with BMDC treated with CpG alone showed no effect. In contrast to BALB/c mice, C57BL/6 mice vaccinated with BMDC pulsed with lysate in the absence of CpG treatment also showed a reduction in lesion development, when compared with non-vaccinated mice, but this effect was less pronounced than that induced by antigen-pulsed BMDC further matured by CpG ODN treatment (FIG. 5A). When the parasite load of the different vaccination groups was analyzed, a striking correlation with the clinical outcome was observed. The parasite numbers of mice with non-protective treatment were similar to those of the control (FIG. 5B). Mice vaccinated with lysate-pulsed BMDC showed a 10-fold reduction in the parasite load. Most notably, those mice vaccinated with CpG-matured lysate-pulsed BMDC had approximately 100-fold less parasites in the footpads (FIG. 5B). These results demonstrate that vaccination with CpG-matured lysate-pulsed BMDC induces a significant protective effect, with a reduction of the parasite load at the site of infection, in both BALB/c and C57BL/6 mice.

### Example 7

### Resistance Induced by CpG-Matured/Lysate-Pulsed BMDC Immunization is Solid and Protects Against Re-Infection

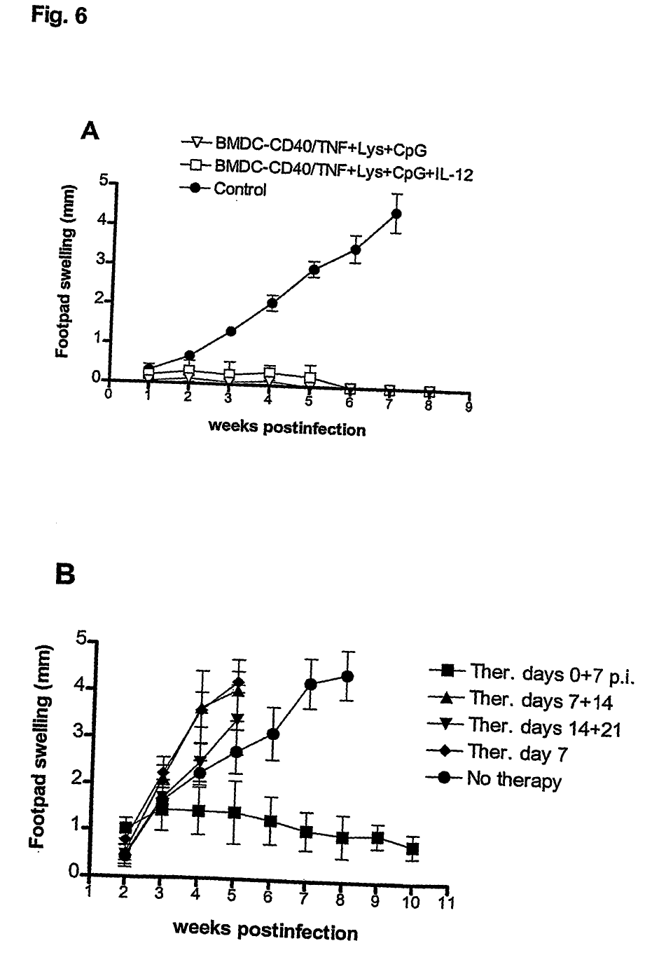

Because of the exceptional efficacy of CpG-matured/lysate-pulsed BMDC in mediating protection (total cure of 100% of the mice, FIG. 1), it was investigated whether the mice that resolved the primary infection were able to resist a second challenge with parasites. To this end, the 10 mice that completely cured in the experiment shown in FIG. 1A were rechallenged with 0.5×10⁶ metacyclic parasites (2.5-fold more than the primary infectious dose) 10 weeks after the first challenge. The results in FIG. 6A show that solid immunity was established by immunization with these BMDC, since the swellings developed after secondary infection was even lower than those after primary challenge. Rechallenged mice showed an almost unreadable footpad swelling (less than 0.5 mm) and most of them completely cured after 3 weeks after the secondary infection. This group of mice was followed up for more than 20 weeks after secondary challenge without any sign of disease.

### Example 8

### The Immunotherapeutic Effect of CpG-Matured/Lysate-Pulsed BMDC is Dependent on the Time of BMDC Administration

Given the unusual potency of these cells in inducing a long-lasting protective Th1 immune response, the next question to address was whether it is possible to cure an already established Leishmania infection. For this purpose, a series of experiments was designed in which naive mice were infected and subsequently treated with CpG-matured/lysate-pulsed BMDC at different time points. The results shown in FIG. 6B indicate that under our experimental conditions, in a very limited time window of not longer than 7 days after infection, it is still possible to redirect the immune response towards a protective phenotype. When mice are treated on day 0 (1 hour post infection) and one week after infection, a very clear therapeutic effect is observed. When therapy is performed one and two weeks after infection the effect is reverted, since not only no curative effect is observed but also the treatment seems to be exacerbating. A similar curve of disease progression is observed when a single therapeutic dose is injected one week after infection, indicating that the therapeutic efficacy exerted by the schedule 0+7 days p.i. is more dependent on the first dose than the second, and strongly challenges the use of this approach at least in this model and under our experimental conditions. Finally, two therapeutic doses on days 14 and 21 p.i. did not show any effect as evidenced by a kinetic in disease progression comparable to that from the control group (FIG. 6B). Thus, a therapeutic application of BMDC in murine leishmaniasis is possible. However, the time of administration seems to be critical for efficacy.

### Example 9

### CpG-Matured/Lysate-Pulsed BMDC Express IL-12

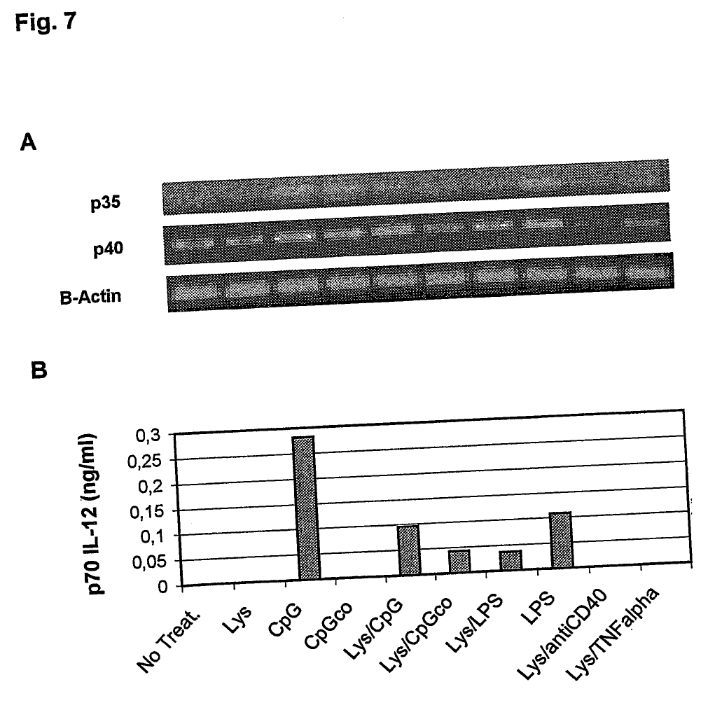

To explore the mechanisms involved in the activation of the protective Th1-like immune response observed in mice vaccinated with CpG-matured/lysate pulsed BMDC, the expression of IL-12 was analyzed. This cytokine is formed by the subunits p40 and p35 and is known to play a key role in the development of Th1 cells. For this purpose, BMDC were treated as described in Example 1, and after 36 hours total RNA was isolated and levels of IL-12 p35 and p40 mRNAs were determined by RT-PCR. Supernatants of the same cultures were also collected and the active p70 form of the protein was measured by ELISA. As shown in FIG. 7A, p40 and p35 mRNA were differentially regulated by pulsing/maturation stimuli in BMDC. CpG and non-CpG ODN as well as LPS induce a very strong up-regulation of p35 mRNA while parasite antigen pulsing down-regulates both basal and induced expression. Among the groups having been treated by pulsing and maturation stimuli, BMDC-CPG combination showed the maximal p35 mRNA level. In contrast to p35, basal levels of the p40 mRNA were apparently unchanged by pulsing only. However, except for lysate-CpG combination, pulsing down-regulated the inducible expression (see LPS and CpGco alone versus LPS-Lys and CpGco-Lys, respectively). Again, among groups having been treated by pulsing and maturation stimuli, BMDC-CpG combination showed the maximal p40 mRNA level. Active p70 protein levels in supernatants were also dependent on the pulsing/maturation treatment as shown in FIG. 7B. As expected, maximal levels of p70 subunit were induced by CpG ODN treatment of BMDC. No p70 IL-12 was detectable in non-treated, pulsed only, and CD40 or TNF-alpha-matured pulsed cultures. However, in spite that again pulsing down-regulated the LPS- and CpG-induced p70 production, once more the last one was the treatment exhibiting the maximal level of functionally active p70 IL-12 among pulsed/matured groups (at least doubling amounts). All these results suggest that the protective effect observed when susceptible mice are vaccinated with BMDC pulsed with Leishmania lysate and matured with CpG ODN, is due to the induction of a strong Th1 immune response, which is correlated with the ability of these cells to secrete active p70 IL-12.

1. A not naturally occurring dendritic cell (DC) having specific antigen presentation properties in an individual comprising a specific disease related antigen and a CpG molecule, wherein said DC derives from CD34⁺ bone marrow precursor cells or peripheral blood monocyte preparations.

2. A DC according to claim 1, wherein the CpG oligonucleotide comprises the nucleic acid sequence 5′-TTCATGACGTTCCTGATGCT-3′

3. A DC according to claim 1, wherein said DC was obtained by prolonged exposure to IL-4 and GM-CSF in vitro.

4. A DC according to claim 1, wherein the antigen is a microbial or a cancer antigen.

5. A DC according to claims 4, wherein the microbial antigen derives from a parasite.

6. A DC according to claims 5, wherein the parasite is Leishmania.

7. A DC according to claims 4, wherein the specific antigen presentation property comprises a Th1 type immune stimulatory response.

8. A pharmaceutical composition comprising a DC of claim 1, optionally together with a pharmaceutically acceptable carrier, diluent and excipient.

9. A pharmaceutical composition according to claims 8 comprising a cytokine.

10. A vaccine comprising a DC according to claim 1 and an additional adjuvant.

11. Use of DC according to claim 1, for the preparation of a medicament for the prophylactic or therapeutic treatment of infectious and cancerous diseases.

12. A method of producing a DC, comprising the following steps: (a) exposing a specific antigen to the isolated DC as defined to any of the claim 1, and (b) treating the DC with one or more CpG oligonucleotides

13. The method of claim 12, wherein the DC derives from mobilized stem cells and is isolated from peripheral blood, wherein said precursor cells have been cultured under conditions allowing to generate functional DCs.

14. The method of claim 12, wherein step (a) and (b) are carried out simultaneously.

15. A pharmaceutical kit comprising several packages, wherein a first package contains DC obtained from CD34⁺ bone marrow precursor cells or peripheral blood monocyte preparations by culturing the cells in IL-4 and GM-CSF, a second package with a CpG molecule and a third package with a disease related antigen.
